(19) United States
(12) Patent Application Publication
(10) Pub. No.: US 2005/0131370 A1
(43) Pub. Date: Jun. 16, 2005

(54) Individually wrapped personal care absorbent articles

(75) Inventors: Richard J. Hantke, Appleton, WI (US); Peter Gebhardt, New London, WI (US); Jennifer E. Pozniak, Menasha, WI (US); Steven J. Nielsen, Conway, AR (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 10/732,821

(22) Filed: Dec. 10, 2003

(51) Int. Cl. A61F 13/15; A61F 13/20

(52) U.S. Cl. 604/385.02

(57) ABSTRACT

An individually wrapped absorbent article package includes an absorbent article placed in a wrapper material folded into a pouch. The pouch may have an opening flap. The wrapper material includes a nonwoven material in at least a border region thereof such that upon the wrapper material being folded into the pouch, portions of the border region of nonwoven material are facing and define a seal zone along sides of the pouch. A frangible seal is formed in the seal zone between the facing borders of nonwoven material. The seal has a seal strength of between about 20 grams-force and about 60 grams-force and an opening noise level of less than about 60 db.

### FIELD OF THE INVENTION

The present invention relates generally to the field of disposable absorbent articles such as feminine care articles, and more particularly to an improved pouch configuration for such articles.

### BACKGROUND

Disposable absorbent articles for absorbing any manner of bodily fluids or exudates are well know, and include, for example, incontinence devices, feminine care articles such as sanitary napkins, panty liners, and so forth. For explanation purposes only, the invention is described as it relates to feminine care articles used to absorb menses and other body fluids typically during a women's menstrual cycle. In addition, such articles may also be used between menstrual cycles for light incontinence purposes.

Since many of these articles are carried in a woman's purse or pocket prior to use, it is advantageous to individually wrap each article to keep it clean and sanitary. By individually packaging each absorbent article, the manufacturer and user can be assured that the article will not become contaminated by the contents of the user's purse, pocket, etc.

Conventionally, the article wrapper consists of one or more layers of a thin sheet or film of thermoplastic material, such as polyethylene, which is folded around the absorbent article and then sealed by the use of heat and/or pressure, ultrasonics, or an adhesive to form a package or pouch. The package is designed to be opened by breaking or tearing the material at or adjacent a seal in order to subsequently remove the absorbent article. Conventional packages are also typically designed so that a soiled article can be wrapped up in the opened package for later disposal.

Most women value their personal privacy and prefer not to advertise to others that they are carrying or using feminine care products. For some individuals, the “public” use of such products can be an anxious and often traumatic experience. Unfortunately, conventional wrapping materials and package designs may only exacerbate this problem. Conventional films used in many absorbent wrappers are inherently “loud” when manipulated. The material “crinkles” when shaken or moved and is particularly loud when the package is opened and the absorbent article is removed, especially if the article is adhered to the inside of the wrapper. Often, the exercise of locating the article in a purse or carry bag is announced to those in relatively close proximity by the sound of the package once it is located and removed. The same situation applies to opening the package. Thus, the consumer's ability to discreetly and quietly store and open the absorbent article is hampered by the packaging materials.

The noise generated in opening conventional pouches is also a function of the seal strength of the seals used to form the pouch. In general, the noise level increases with seal strength because more energy is required to break the seal, resulting in a relatively loud “zippering” noise. However, seal strength is often dictated by the requirements of modern high speed processing lines. For example, the pouches are typically handled and compressed between conveyors and belts before final packaging. As the pouches are compressed, trapped air within the pouches can create substantial internal pressure. If the seals are not sufficiently strong, the internal pressure will burst the seals resulting in defective products.

Packaged absorbent articles are commercially available wherein the articles are contained in sealed pouches made substantially entirely of a nonwoven material. Such commercial articles are known in Japan from Unicharm company under the “Sofy” brand name (the particular absorbent pad known as “Shikkari Kyushu Gurard Yoru-yo Super”), and in Korea from Yuhan-Kimberly company under the “KOTEX Good Feel’ brand name.

The present invention relates to an improved pouch for individually wrapped absorbent articles that incorporates a nonwoven material and can be readily manufactured and quietly stored, carried, and opened.

### SUMMARY

Objects and advantages of the invention will be set forth in part in the following description, or may be obvious from the description, or may be learned through practice of the invention.

The present invention relates to a unique wrapper or pouch configuration for individually packaged absorbent articles, for example feminine care articles such as sanitary pads, liners, tampons, and so forth. It should be appreciated that the invention is not limited to any particular type of absorbent article. The package includes a wrapper material folded into a pouch, with the absorbent article carried within the pouch for subsequent retrieval by the consumer prior to use. The pouch has at least one sealed seam, for example along one or more edges of the pouch, or set in from the edges, that is opened by a consumer to retrieve the absorbent article. The sealed seam is defined by thermally sealed facing regions of a nonwoven material. The seam is formed as a function of the particular properties of the nonwoven material such that the seam has a strength balanced between having a strong enough seal to process the pouches in a modern high speed processing line, and having a light or “weak” enough seal to open the pouch easily and quietly. In a particular embodiment, the seal strength is between a minimum average load value of about 20 grams-force to ensure adequate processing strength, and a maximum average load value of 60 grams-force to minimize opening noise by a consumer breaking and opening the seal. Preferably, the noise generated by the opening process is less than about 60 dbA. Due to its permeability, the nonwoven material also ensures that air entrapped within the pouch may escape through the material during the various manufacturing processes. Accordingly, it should be ensured that sufficient nonwoven material is exposed to the interior volume of the pouch as a function of the permeability of the particular type of nonwoven material selected.

It should be understood that the term “seal” is meant to encompass all types of pouch opening configurations, including sealed side seams with an opening flap, perforated tear lines, scored lines, tear zones, etc.

It should be appreciated that the invention is not limited to any particular type of pouch configuration, or number and type of absorbent article carried with the pouch. In a relatively simple embodiment, the pouch is formed by folding the wrapper material in half so as to align the lateral sides and opposite longitudinal ends of the material. The sides and open end of the pouch are then sealed with one or more absorbent articles inside. In a particularly well known flap-type pouch configuration, the wrapper material is folded at a first fold axis at one end, and at a second fold axis at the opposite end such that aligned lateral sides of the wrapper material are brought together to define the pouch. The end folded at the second fold axis defines an opening flap that is grasped and pulled generally away from the pouch by the consumer to open the pouch and retrieve the absorbent article.

The use of the unique nonwoven material seam according to the invention provides a “quieter” seam as compared to conventional film pouches, particularly at the flap seams. With conventional film pouches, the inherent “loudness” is a result of the characteristics of the film material and is particularly enhanced along the seams where film material is bonded to film material resulting in thicker and stiffer seams. Thus, the process of breaking or separating the flap seams to open the pouch is a relatively noisy and distinct event. The noise associated with the seams and the opening process in general is reduced with pouches according to the present invention. The pouch seams are more pliant and thus less noisy, and the flap seams are separated to open the pouches with far less noise. Also, the level (decibel) of noise not withstanding, the type of noise generated with the configuration according to the present invention is different from and far less recognizable than the distinct type of noise generated from film material seals.

In a particular embodiment of a flap-type pouch according to the invention, the lateral sides of the folded-over flap are sealed with the sides of the pouch. The leading edge of the flap may be unsealed between the sealed lateral sides such that a user may easily grasp the flap at the unsealed leading edge for opening the pouch. The flap may extend at least partially over a front panel of the pouch and be sealed with the lateral sides of the front panel as well.

The regions of nonwoven material used in forming the sealed seams may be provided in various ways. In one particular embodiment, the wrapper material is formed substantially entirely of a sheet of one or more layers of a nonwoven material, such as a spunbond or meltblown material, or a spunbond-meltblown-spunbond material. With this embodiment, air entrapped within the pouch may readily escape through any surface of the pouch.

In an alternate embodiment, the wrapper material combines the benefits of a film pouch with the benefits of a nonwoven material seam. For example, the wrapper material may be a composite of a first sheet of nonwoven material suitable for forming the desired type of seal with respect to strength and noise, and an additional sheet or layer of a film material attached to the nonwoven material. For example, the film material may be laminated to the nonwoven material. The film material sheet has dimensions such that a border region of exposed nonwoven material is defined along at least one edge of the composite material to form the sealed seam, for example along the lateral sides of the composite material. Alternately, the nonwoven material may frame the film material such that border regions of the nonwoven material are defined along the lateral and longitudinal sides of the composite material. The film material is desirably configured such that, upon folding the wrapper material into the pouch, the film material essentially forms a partial film liner within the pouch yet leaves at least a portion of the nonwoven material exposed to allow entrapped air to escape from the pouch during the manufacturing processing. The exposed portion may be defined, for example, adjacent to the border region used to form the sealed seam. In an alternate embodiment, the nonwoven material may be exposed through holes or passages defined through the film material, and so forth. Alternately, the film material may be air-permeable. The film material may be any one or combination of well-known suitable film materials, and is desirably impervious to liquids and vapor permeable.

It should be appreciated that various suitable bonding processes may be used to seal the nonwoven material along the sealed seams of the pouch. For example, a conventional heated bonding roll may be used to thermally seal the nonwoven material. The bonding parameters, such as temperature, dwell time, etc., may be readily empirically determined by those skilled in the art as a function of the type of nonwoven material, processing speed, desired seal strength, and so forth.

Aspects of the invention will described below in greater detail by reference to particular embodiments, examples of which are illustrated in the figures.

### BRIEF DESCRIPTION OF THE FIGURES

FIG. 1 is a perspective view of a wrapped absorbent article package according to the invention.

FIG. 2 is a perspective view of the package of FIG. 1 in an opened condition.

FIG. 3 is a perspective view of an alternate embodiment of a package according to the invention prior to sealing the wrapper material to form a pouch.

FIG. 4 is a perspective view of another embodiment according to the invention prior to sealing the wrapper material to form a pouch.

FIG. 5 is a perspective view of still a different embodiment according to the invention prior to sealing the wrapper material to form a pouch.

FIG. 6 is a perspective view of an alternate embodiment of a wrapped absorbent article package according to the invention.

FIG. 7 is a perspective view of the testing apparatus used to evaluate various materials and products, with the apparatus door open.

FIG. 8 is a perspective view of the testing apparatus used to evaluate various materials and products, with the apparatus door closed.

FIG. 9 is a plan view of the apparatus of FIG. 7 taken along arrow 190.

FIG. 10 is an alternative perspective view of the testing apparatus used to evaluate various materials and products, with portions broken away to show underlying features.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Referring to the figures, embodiments of an individually wrapped absorbent article package 10 are generally illustrated. In particular, an absorbent article 12 is carried in the interior volume 24 of a pouch 22. It should be understood that any number of articles 12 may be carried within a single pouch 22. The pouch 22 is formed from a wrapper material 14, as described in detail below. It should be appreciated that the invention is not limited to any particular type of absorbent article 12. For example, the absorbent article 12, may be a personal hygiene article such as a sanitary napkin, a panty liner, a labial pad, a tampon, an incontinence pad, or any other type of absorbent article which can be used to absorb menstrual fluid, urine, body fluid, body exudates, etc. A detailed description of such conventional absorbent articles is not necessary for purposes of the present invention. For purposes of describing the invention only, the absorbent article 12 is shown and referred to herein as a feminine care sanitary pad or napkin. The absorbent article 12 may be folded in any desired pattern to fit in the package 10. For example, the absorbent article 12 may be folded in half, or in a tri-fold pattern, as with many conventional folded and wrapped articles known in the art.

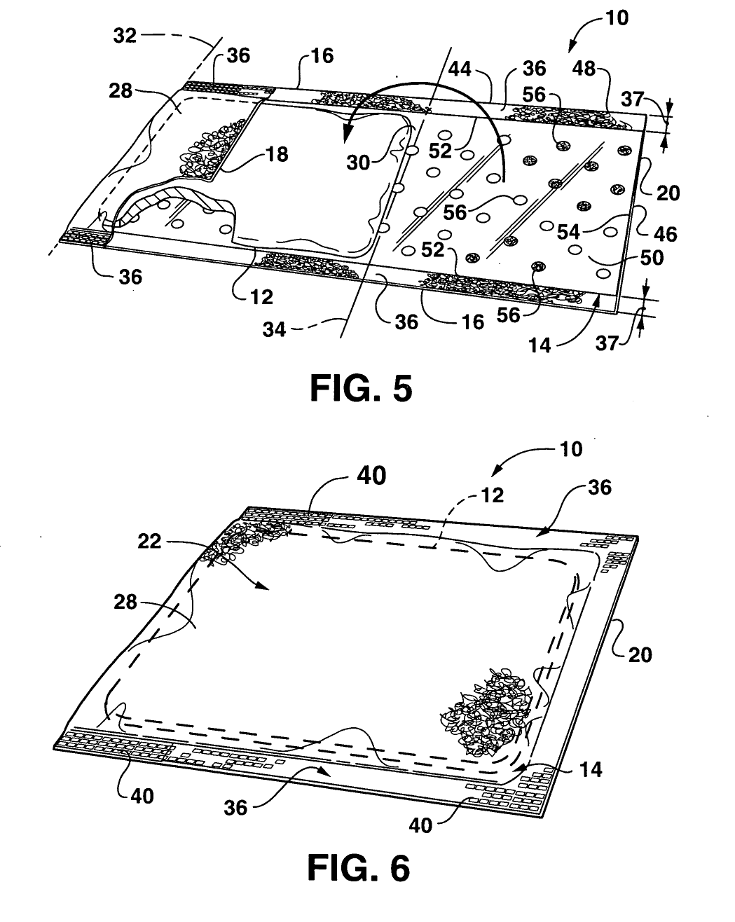

The package 10 includes an elongate piece of wrapper material, generally 14, folded and sealed into the pouch configuration 22. For example, referring to FIGS. 2 and 5, the wrapper material 14 may be an elongated rectangular piece having a first longitudinal end 18, an opposite second longitudinal end 20, and generally parallel lateral sides 16 extending between the ends 18 and 20.

The invention is not limited to any particular type of pouch configuration. Various pouch configurations are known and used in the art for individually packaging feminine care absorbent articles, and any such configuration may be used in a package 10 according to the invention. The unique features of the present wrapper material 14 will provide a benefit to any pouch configuration. In the illustrated embodiment, the pouch 22 is similar to the pouch configuration used for Kotex® Ultrathin pads from Kimberly-Clark Corporation.

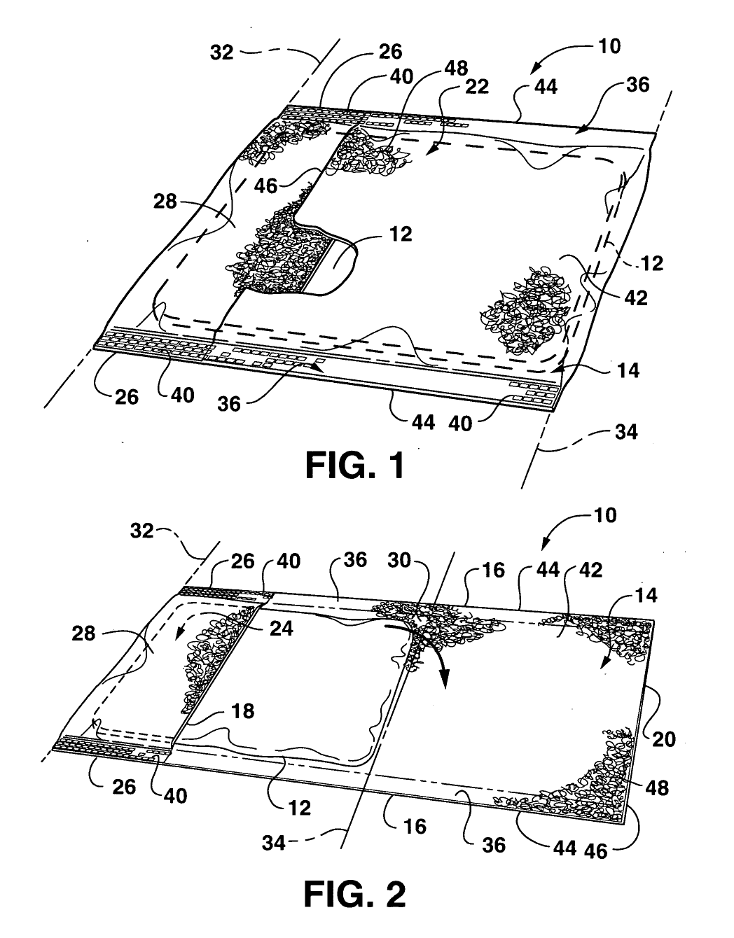

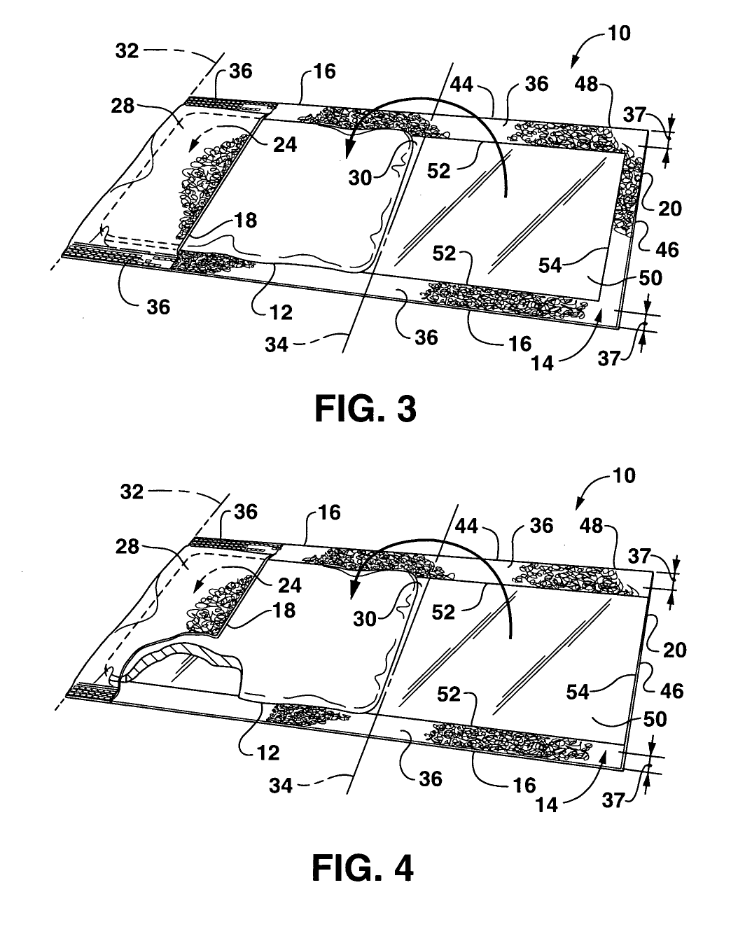

FIG. 2 is a view of the pouch 22 of FIG. 1 after it has been opened. From this figure (and FIGS. 3 through 5), it can be seen that the wrapper material 14 is essentially folded around the absorbent article 12 such that the pouch 22 is formed around the article. In the particular flap-type pouch embodiments illustrated in FIGS. 1 through 5, the wrapper material 14 is first folded at a first fold axis 32 such that the first end 18 is folded over the absorbent article 12 towards but spaced from the second end 20 to define at least a portion of a front panel 28 of the pouch 22. The distance between the first end 18 and second end 20 may vary depending on the length of the absorbent article 12 and desired length of a resulting flap 42, as described in greater detail below.

Once folded, the aligned lateral sides 16 of the wrapper material 14 define lateral edges 26 of the pouch 22. The second end 20 of the wrapper material 14 is then folded at a second fold axis 34 so as to extend at least partially back over the first end 18 and thus defines the opening flap 42 that closes off the pouch 22, as particularly seen in FIG. 1. The portion of the wrapper material 14 between the fold lines 32 and 34 define a back panel 30 of the pouch 22. The flap 42 has lateral edges 44 that align with the material sides to complete the lateral side edges 26 of the pouch 22. The sides of the package 10, including the flap lateral edges 44 are bonded along a seal zone 40 having a width 37 as indicated in FIGS. 3 through 5 in a conventional manner, for example in a simultaneous heat/pressure embossing roll procedure. The seals are “frangible” seals in that the sealed layers will separate or pull apart upon a user opening the pouch 22.

The term “seal zone” should be understood to be any region of the wrapper material 14 wherein seals are made to define the pouch 22. For example, the seal zones 40 may extend along and include the lateral sides of the wrapper material 14, or may be set in from the lateral sides. All such configurations are within the scope and spirit of the invention.

Referring to FIG. 1, the flap 42 has a leading edge 46 that may be unbonded to the front panel 28 between the seal zones 40 so that a consumer may easily slide a finger or other object below the edge 46 to open the pouch 22 by pulling the flap 42 away from the pouch such that the seals along the seal zones 40 are broken. In an alternate embodiment, the flap 42 may be sealed completely across the leading edge 46.

As described, the pouch 22 has at least one thermally sealed seam in one of the seal zones 40 that is opened by the user to retrieve the article 12. According to the embodiments of the invention, this seal is formed between facing regions of a nonwoven material provided at least in the seal zones 40. Because the seal is formed directly between opposing layers of nonwoven material, the seal is inherently “quieter” than film seals upon being opened. The seam is formed as a function of the particular properties of the nonwoven material such that the seam has a seal strength balanced between a strong enough seal to process the pouch 22 in a modern high speed processing line, and having a light enough seal to open the pouch 22 easily and quietly. In a particular embodiment, the seal strength is between a minimum average load value of about 20 grams-force to ensure adequate processing strength, and a maximum average load value of 60 grams-force to minimize opening noise by a consumer breaking and opening the seal. Preferably, the noise generated by the opening process is less than about 60 db.

The regions of nonwoven material used in forming the sealed seams may be provided in various ways. For example, in one particular embodiment, the wrapper material 14 is formed substantially entirely of a sheet 48 of one or more layers of a nonwoven material, such as a spunbond or meltblown material, or a spunbond-meltblown-spunbond material. With this embodiment, air entrapped within the pouch 22 during the manufacturing process may readily escape through any surface of the pouch 22.

In alternate embodiments, the wrapper material 14 combines the benefits of a film pouch with the benefits of a nonwoven material seam. For example, referring to FIGS. 3 through 5, the wrapper material 14 may include a first sheet of nonwoven material 48 suitable for forming the desired type of seal with respect to strength and noise, and an additional sheet or layer of a film material 50 attached to the sheet of nonwoven material 48, the film material having lateral sides 52 and longitudinal ends 54. The film material 50 may be any one or combination of well-known suitable film materials, and is desirably impervious to liquids and vapor permeable.

In one particular configuration, the film material 50 may be laminated to the nonwoven material 48 and is desirably configured such that, upon folding the wrapper material 14 into the pouch 22, the film material 50 essentially forms a partial film liner within the pouch 22 yet leaves at least a portion of the nonwoven material 48 exposed to allow entrapped air to escape from the pouch 22 during the manufacturing processing. Alternately, the film layer may have sufficient air permeability such that it is not necessary that the nonwoven material be exposed beyond the seal zones 40. The film layer 50 and nonwoven material layer 48 may be laminated together by any lamination technique known to those skilled in the art. Suitable lamination means include, but are not limited to, adhesives, ultrasonic bonding and thermo mechanical bonding as through the use of heated calendaring rolls. Such calendaring rolls will often include a patterned roll and a smooth anvil roll, though both rolls may be patterned or smooth and one, both or none of the rolls may be heated. The figures illustrate an aesthetic pattern defined in the laminated wrapper material 14.

In particular embodiments utilizing the film material 50, the film sheet may have dimensions such that a border region 36 of the nonwoven material is provided at least partially around the film material sheet 50, for example along the lateral sides 16 of the composite wrapper material 14. Referring to FIGS. 3 and 4, it can be seen that the border regions 36 have a width greater than the width 37 of the seal zones 40. Thus, the exposed portion of the nonwoven material is defined by a portion of the border regions 36 that extend inwardly of the seal zones 40. In the embodiment of FIG. 3, the nonwoven material sheet 48 frames the film material 50 such that border regions 36 of the nonwoven material are defined along the lateral sides 16 and longitudinal ends 18, 20 of the composite wrapper material 14, as shown for example in FIG. 3.

In the embodiment of FIG. 4, the longitudinal ends 54 of the film material sheet 50 are coincident with the longitudinal ends 18, 20 of the nonwoven material 48 such that the border regions 36 of nonwoven material are formed only along the lateral sides 16 of the composite wrapper material 14. This embodiment may be desired from a manufacturing standpoint in that a continuous strip of the composite wrapper material may be simply cut into desired lengths to form the pouches 22.

In an alternate embodiment depicted for example in FIG. 5, the nonwoven material may be exposed through holes or passages 56 defined through the film material sheet 50, and so forth. In this embodiment, it is not necessary for the lateral sides 52 of the film material sheet 50 to extend inwardly of the width 37 of the seal zones 40.

It should be appreciated that various suitable bonding processes may be used to seal the nonwoven material along the seal zones 40 of the pouch 22. For example, a conventional heated bonding roll may be used to thermally seal the nonwoven material. The bonding parameters, such as temperature, dwell time, etc., may be readily empirically determined by those skilled in the art as a function of the type of nonwoven material, processing speed, desired seal strength, and so forth. Also, the bonding pattern may be any one or combination of suitable patterns, the checkered pattern illustrated in the figures being for purposes of illustration only.

FIG. 6 illustrates an embodiment wherein the pouch is formed simply by folding the wrapper material 14 in half and sealing the nonwoven material in the seal zones 40 along three sides of the pouch. The nonwoven material is provided in border regions 36 sufficient for forming suitable seals 40. Any of the film material sheets 50 described above may be used with this embodiment as well. The embodiment of FIG. 6 does not utilize an opening flap, and the user opens the pouch 22 by grasping the front and back panels and pulling the panels apart until at least one of the seams 40 separates. In this embodiment, the seal is defined between opposite opposing layers of wrapper material 14. In an alternate embodiment (not illustrated), the seal may be defined as a perforated or scored line in a single layer of the wrapper material 14. All such configurations are within the scope and spirit of the invention.

The formation of films 50 useful with the present invention is well known to those of ordinary skill in the art and need not be discussed herein in detail. One type of film that may be used is a nonporous, continuous film that, because of its molecular structure, is capable of forming a vapor-pervious barrier. Among the various polymeric films which fall into this category include films made from poly(vinyl alcohol), polyvinyl acetate, ethylene vinyl alcohol, polyurethane, ethylene methyl acrylate, and ethylene methyl acrylic acid to make them breathable. If desired, it is also possible to add fillers to the film such as, for example, calcium carbonate and titanium dioxide, to increase opacity, decrease cost, and create a breathable film if the filled film is subsequently stretched. If the film layer is not sufficiently thin, then it may be necessary to further thin the film by stretching it in an apparatus such as a machine direction orienter (MDO) unit. An MDO has a plurality of stretching rollers which progressively stretch and thin the film in the machine direction (direction of travel of the film through the machine).

Another type of film which may be useful is an air permeable microporous film. These films have a number of interconnecting voids or holes which provide pathways for the transportation of water molecules from one surface to another. The passageways are sufficiently small so that only vapors and not fluids can pass through them.

The nonwoven component 48 of the wrapper material 14 may be any one or combination of suitable fibrous materials. As used herein, the term “fiber” or “fibrous” refers to elongated individual natural or synthetic strands (as compared to a continuous film layer). Synthetic fibers are formed by passing a polymer through a forming orifice such as a die. Unless noted otherwise, the terms “fibers” or “fibrous” include discontinuous strands having a definite length and continuous strands of material, such as filaments. The fibrous material may comprise any one or combination of non-woven or woven materials and is intended to give the pouch a soft and cloth-like tactile feel and to dampen and reduce noise associated with storing, carrying, and opening the pouches 22. Non-woven materials may be preferred from a manufacturing standpoint. However, woven materials, including any manner of synthetic or natural cloth, are within the scope and spirit of the invention.

As used herein the term “nonwoven” material means a web having a structure of individual fibers or threads which are interlaid, but not in an identifiable manner as in a knitted fabric. Nonwoven fabrics or webs have been formed from many processes such as for example, meltblowing processes, spunbonding processes, bonded carded web processes, etc. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

The nonwoven material 48 may comprise a non-woven meltblown web. Meltblown fibers are formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al. Generally speaking, meltblown fibers may be microfibers that may be continuous or discontinuous, are generally smaller than 10 microns in diameter, and are generally tacky when deposited onto a collecting surface.

The nonwoven material 48 may comprise a non-woven spunbond web. Spunbonded fibers are small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel. et al., U.S. Pat. No.3,692,618 to Dorschner, et al., U.S. Pat. No.3,802,817 to Matsuki, et al., U.S. Pat. No.3,338,992 to Kinney, U.S. Pat. No.3,341,394 to Kinney, U.S. Pat. No.3,502,763 to Hartman, U.S. Pat. No.3,502,538 to Levy, U.S. Pat. No.3,542,615 to Dobo, et al., and U.S. Pat. No.5,382,400 to Pike, et al. Spunbond fibers are generally not tacky when they are deposited onto a collecting surface. Spunbond fibers can sometimes have diameters less than about 40 microns, and are often between about 5 to about 20 microns.

The nonwoven material 48 may comprise a spunbond/meltblown/spunbond, or SMS, material. A typical SMS material is described in U.S. Pat. No. 4,041,203 to Brock et al. Other SMS products and processes are described for example in U.S. Pat. Nos. 5,464,688 to Timmons et al.; U.S. Pat. No.5,169,706 to Collier et al.; and U.S. Pat. No.4,766,029 to Brock et al. Generally, an SMS material will consist of a meltblown web sandwiched between two exterior spunbond webs. Such SMS laminates have been available commercially for years from Kimberly-Clark Corporation under marks such as Spunguard® and Evolution®. The spunbonded layers on the SMS laminates provide durability and the internal meltblown layer provides porosity and additional cloth-like feel.

Suitable non-woven web materials 48 may also be made from bonded carded webs and airlaid webs. Bonded carded webs are made from staple fibers which are sent through a combing or carding unit, which separates or breaks apart and aligns the staple fibers to form a nonwoven web. Once the web is formed, it then is bonded by one or more of several known bonding methods.

Airlaying is another well known process by which fibrous webs can be formed. In the airlaying process, bundles of small fibers having typical lengths ranging from about 6 to about 19 millimeters are separated and entrained in an air supply and then deposited onto a forming screen, usually with the assistance of a vacuum supply. The randomly deposited fibers then can be bonded to one another using known bonding techniques.

Having described certain specific embodiments of the present invention, a series of sample pouches with a pad inside were made and tested to further illustrate the present invention. The results of these tests, and the test procedures used, are set forth below.

### TEST PROCEDURES AND EXAMPLES

Pouches made in accordance with aspects of the invention were tested as set forth below. The pouches were tested as three different Code groups:

Code A—Nonwoven (NW) Pouch—Low Seal Strength: Pouches constructed in accordance with FIGS. 1 and 2 from a SMS material having a basis weight of 0.8 osy (polypropylene Spunbond and Meltblown components; 70 to 75% of the material being the Spunbond component). The sides and flap were sealed in a grid pattern as illustrated in FIGS. 1 and 2 with a seal bar temperature of 220° F.

Code B—Nonwoven (NW) Pouch—Medium Seal Strength: Same as Code A except with a seal bar temperature of 270° F.

Code C—Nonwoven (NW) Pouch—High Seal Strength: Same as Code A except with a seal bar temperature of 310° F.

The pouch Codes were tested against three commercially available products:

Kotex® pad film pouches from Kimberly-Clark Corp.

“Kotex-Good Feel” nonwoven pouches from Yuhan-Kimberly (Korea)

“Sofy” nonwoven pouches from Unicharm (Japan)

A. Sound Testing Apparatus and Procedure

A Quiet Test Chamber (QTC) was developed in order to quantify noise generation from disposable absorbent products and component assemblies.

1. Apparatus

Each of the materials in the examples that follow was tested in a testing apparatus comprised of a test chamber, a control chamber, and a sound level meter. The purpose of the apparatus is to manipulate an article in a controlled noise environment, and to accurately quantify the noise produced by the movement of the article. In general terms, a specimen is secured within the testing chamber, and stretched and relaxed repeatedly. The stretching and relaxing causes the specimen to generate noise which is recorded by the sound level meter.

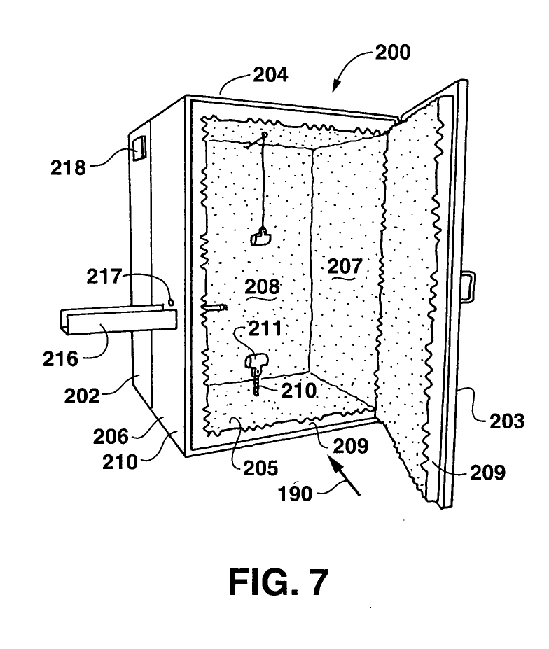

The testing apparatus is illustrated in FIGS. 7-10. The testing apparatus 200 includes a test chamber 201 and a control chamber 202. The test chamber 201 includes a door 203, a top wall 204, a bottom wall 205, two side walls 206 and 207, and a rear wall 208. The door and each wall are constructed of 0.25-inch thick 6061 grade anodized aluminum. The door 203 and rear wall 208 are each 36 inches (91.4 cm) in height and 24 inches (61.0 cm) in width. The test chamber side walls 206 and 207 are each 36 inches (91.4 cm) high and 18 inches (45.7 cm) wide. The test chamber top and bottom panels are each 24 inches wide (61.0 cm) and 18 inches (45.7 cm) long. The interior surface of the door 203 and each wall 204-208 has applied thereto two-inch thick polyurethane sound-dampening foam 209, available from Illbruck Inc., a company having offices in Minneapolis, Minn., under the brand name SONEX and stock number SOC-2. As shown, a sound level meter support 216 extends perpendicularly outward from side wall 206 just below a microphone orifice 217. The microphone orifice 217 is positioned 14.5 centimeters above the floor of the bottom wall 205, and is further centered between the door 203 and the rear wall 208. The sound level meter support 216 is constructed of aluminum and is bolted to side wall 206.

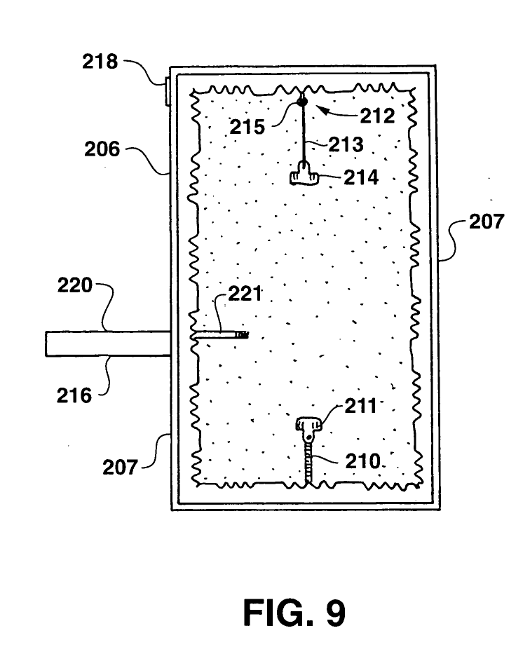

A lower slide bracket 210, a six-inch high Series A1500 Model available from Velmex, Inc., Bloomfield, N.Y., U.S.A., extends from the bottom wall 205 into the test chamber 201, and a lower clamp 211 is affixed to the lower slide bracket 210. An eyelet 212 (FIG. 9) extends from the top wall 204 into the test chamber 201, and a lanyard 213 extends through the eyelet 212. One end of the lanyard 213 extends into the test chamber 201, and has an upper clamp 214 affixed thereto. The other end of the lanyard 213 extends into the control chamber 202 through a lanyard orifice 215, which is 16 millimeters (5/8 inch) in diameter. The lanyard used was a premium-braid, 80-lb test Spiderwire®, part number SB80G-300, manufactured by Johnson Worldwide Associates (JWA), Inc., a company having offices in Racine, Wis., U.S.A. Both the lower and upper clamps are two inches wide and were purchased from Tri County Machining, Inc., Appleton, Wis., U.S.A., model 11220.

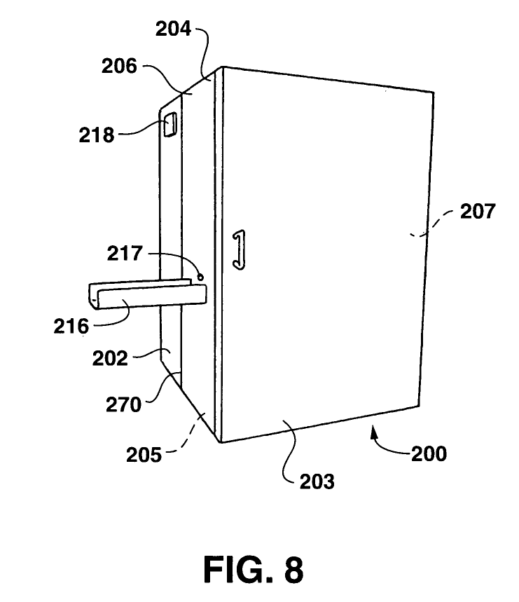

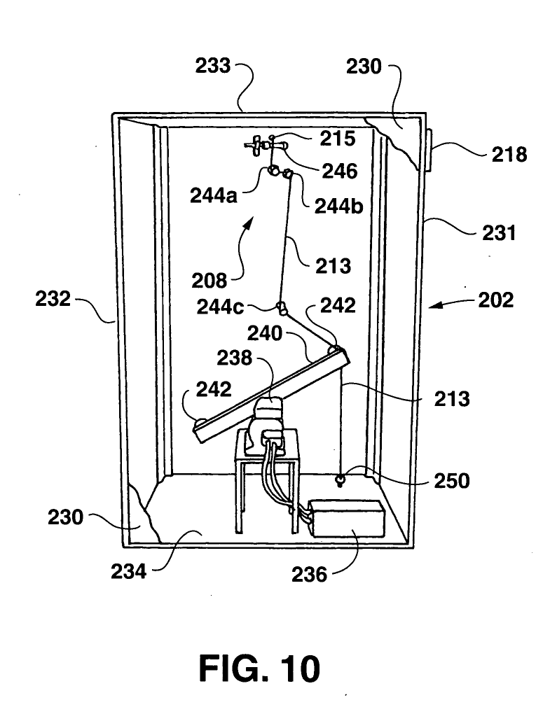

As shown in FIG. 10, the control chamber 202 includes a front wall 230, two side walls 231 and 232, a top wall 233, and a bottom wall 234. Each wall is constructed of 0.125-inch thick 6061 grade anodized aluminum. The front wall 230 is 36 inches (91.4 cm) high and 24 inches (61.0 cm) wide. The control chamber side walls 231 and 232 are each 36 inches high (91.4 cm) and 12 inches (30.5 cm) wide. The control chamber top and bottom walls 233 and 234 are each 24 inches (61.0 cm) wide and 12 inches (30.5 cm) long. The control chamber 202 is bolted (not shown) to the outer surface of rear wall 208 along seam 270 (FIG. 8). The outer surface of the rear wall 208, and the front wall 230, two side walls 231 and 232, top wall 233, and bottom wall 234 of the control chamber 202 are each coated with 0.600-inch thick sound insulating material, part number NYC-600BE, available from Small Parts, Inc., a company having offices in Miami Lake, Fla., U.S.A. The control chamber 202 houses a power supply 236 and a brushless motor 238. The power supply 236 is a 24 volt DC power supply rated for 1.25 amps at 30 watts, model number MSCA-0305, available from Astrodyne Corp., a company having offices in Taunton, Mass., U.S.A. The brushless motor 238 is an AXH series DC brushless gear motor with controller, part number AXH230KC-50, available from Oriental Motor USA, a company having offices in Chicago, Ill., U.S.A. The motor 238 and motor controller are mounted on an elevated anodized aluminum pad 239. As shown, the motor 238, powered by power supply 236, rotates bar assembly 240 which has a spool bearing 242 at each end. The central axis of the motor spindle is 11 inches (27.9 cm) above the inward surface of the bottom wall 234. The bar assembly 240 is 15 inches (38.1 cm) long, and the spool bearings' axes are spaced 14.5 inches (38.1 cm) apart.

The lanyard 213, which originates in the test chamber 201, enters the control chamber 202 through orifice 215 in the rear wall 208 and passes over a bearing mandrel 246 whose central axis is positioned 3.5 inches from the rear wall 208. The lanyard then passes over guide rollers 244a-c, around a spool bearing 242, and is affixed to an eyelet anchor 250. The guide roller 244c is positioned 8.5 inches directly above the central axis of the spindle of the motor 238, and the eyelet anchor 250 is positioned 19.5 inches from the central axis of the guide roller 244c. When the power supply 236 is activated and the bar assembly 240 rotates, the spool bearings 242 momentarily displace a portion of the lanyard 213 out of the test chamber 201 into the control chamber 202 in a cyclical manner, providing the movement action required to manipulate the test specimen. During the test procedure, the bar assembly 240 can make a full rotation every four seconds, causing the lanyard 213 and upper clamp 214 to move up and down with the test chamber once every two seconds, or thirty times per minute. For purposes of the present invention, it is only necessary for the upper clamp to move in a single upward stroke to separate the flap member from the pouch. The guide rollers 244a-c, bar assembly 240, spool bearings 242, and eyelet anchor 250 are positioned such that as the bar assembly 240 rotates, the upper clamp 214 travels a total vertical distance of ten centimeters in each direction. It is contemplated that the positions of the guide rollers 244a-c, bar assembly 240, spool bearings 242, and eyelet anchor 250 could be modified by one of skill in the art, so long as the resulting testing apparatus 200 is configured such that the upper clamp 214 travels a total vertical distance of ten centimeters in each direction during operation of the apparatus 200. The control chamber also includes a start/stop control box 218 secured thereto, which is used to activate and deactivate the power supply 236.

The testing apparatus 200 further includes a sound level meter 220 (FIG. 9), such as model 1900, equipped with a model OB-100 octave filter set, both available from Quest Technologies, a company having offices in Oconomowoc, Wis., U.S.A. The sound level meter is supported by a model QC-20 calibrator and QuestSuite master module software, each also available from Quest Technologies. The software is installed on a personal computer (not shown). The sound level meter includes a microphone 221 extending 4.75 inches (12 centimeters) therefrom.

Prior to testing a specimen using the testing apparatus 200, the following steps are followed:
1. Calibrate the sound level meter 220 following the instructions in the manufacturer's manual.
2. Insert the full length of the microphone 221 into the testing chamber 201 (it should extend past the wall and sound dampening material approximately 2.5 inches), positioned at a 90-degree angle to side wall 206. Allow the sound level meter 220 to rest in the sound level meter support 216.
3. Push the Start button on the control box, without a specimen in the clamps 211/214.
4. Set the octave filter to 2 kHz and take a reading each second for five minutes by activating the sound level meter for five minutes. This allows the amount of noise (at 2 kHz) generated by the testing apparatus alone to be quantified. Noise from the apparatus at other frequencies will not be detected.

The phrase “at 2 kHz”, as used when referring to the amount of noise produced during an experiment and as measured using the testing apparatus 200, means the amount of noise provided by sound waves having frequencies from about 1.414 kHz to about 2.828 kHz.

Having calibrated the testing apparatus 200 and having identified the baseline noise generated thereby, a specimen may be testing in the following manner:
5. Position the upper clamp 214, inside the chamber at its fully protracted (lowered) position by, if necessary, momentarily activating the power supply 236 to rotate the bar assembly 240.
6. Position the transverse center portion of the flap of a pouch to be tested within the upper clamp 214.
7. Raise the lower clamp 211 to its highest position on the lower slide bracket 210. Position the transverse bottom center portion the pouch to be tested within the lower clamp 211. Close the door 203.
8. Set the octave filter to 2 kHz and take a reading each second for one minute by activating the sound level meter for one minute.
9. Press the Start button to initiate movement of the apparatus and specimen. The flap seals will be separated essentially immediately upon movement of the upper clamp.
10. Record the sound level meter for 5 seconds.
11. Push the stop button to stop movement of the upper clamp.
12. Repeat steps 5 through 11 for each sample of each code set to be tested.
13. Record the LMAX values of each of the five second tests at the 2 KHz frequency.
14. Download the data to a personal computer, such as per the guidelines in the instruction manual accompanying the QuestSuite master module software, and calculate the mean and standard deviations or each code.

Ambient noise within the test chamber may be determined by closing the door 203 and executing the steps outlined above with the apparatus off at all times and with no specimen in the test chamber.

When the testing apparatus is operated without a specimen, average sound levels of about 25.5 dB (std. dev. ˜0.16) at 2 kHz are recorded within the testing chamber due to the operation of the apparatus itself and other ambient noise. When properly constructed, the testing apparatus should produce no more than 26 dB at 2 kHz when operated without a specimen. When a specimen is tested, the sound level recorded within the chamber is that of the apparatus and the moving specimen combined.

The results of each test are present in the Table below.

2. Definitions

LMax—the maximum level—is the highest sound level recorded during a measurement interval with a particular response level setting (Slow or Fast) and weighting (A or C). LMax is the highest value that is included in LAvg or dose calculations.

Response level (Slow response used for testing)—Also called the response rate or the time constant. Instruments used to measure sound levels have selectable response time constants, which were originally established to describe the dynamic response characteristics of analog sound level meters. The response rate determines how quickly the unit responds to fluctuating noise. The time constants are:
Slow—1 second (1,000 milliseconds)
Fast—0.125 second (125 milliseconds)
Peak—50 microseconds
Impulse—35 millisecond rise with a decay time of 1.5 seconds
   Typical occupational and environmental noise regulations require a Slow response rate.

Weighting (“A” weighting used for tests)—The filtering of sound before averaging. A, B, C, and Linear are the standard weighting networks (circuits) available in noise dosimeters and sound-level meters. These frequency filters cover the frequency range of human hearing. A weighting is the most commonly used filter in both industrial noise applications and community noise regulations. A-weighted measurements are written as dBA or dB(A). The A-weighted filter attempts to make the dosimeter respond the way the human ear hears. This filter attenuates the frequency below several hundred hertz as well as the high frequencies (about 6,000 hertz).

LAvg—The average sound level (in dB) computed for a chosen time duration, based on a 4, 5, or 6 dB exchange rate. LAvg with a 3 dB exchange rate is referred to as LEQ (equivalent sound level). All sound levels at or above the threshold level are averaged into the calculations relating to noise exposure. LAvg is typically calculated with no threshold, with a threshold of 80 dB, and with a threshold of 90 dB.

Exchange Rate—The rate in which sound energy is averaged over time.

3. Sound Level Meter Parameters for Testing:
Range: 30-90 dB
Weighting: “A”
Time Constant: Slow
Threshold: Off
Exchange Rate: 3 dB
Peak Weighting: “C”

4. Results

Peak Sound Level (L_{MAX}) (dbA)

| | | | | | Y-K Korean | Jap. Uni- | |
|---|---|---|---|---|---|---|---|
| | | | | Kotex | Good | Charm | |
| | Code | Code | Code | Film | Feel NW | NW | No |
| | A | B | C | Pouch | Pouch | Pouch | Product |
| 1 | 58.1 | 56.3 | 67.7 | 61.6 | 54.7 | 53.6 | 53 |
| 2 | 59.3 | 58.9 | 68 | 63.7 | 54.6 | 52.4 | 53.1 |
| 3 | 62.6 | 58.6 | 68.2 | 65.1 | 53.1 | 54.1 | 53.2 |
| 4 | 60 | 63.2 | 67.3 | 64 | 55.9 | 53.1 | 54.1 |
| 5 | 58 | 65.2 | 67 | 63.2 | 53.3 | 53.2 | 53.6 |
| 6 | 58.4 | 60.4 | 65.8 | 62.6 | 55.8 | | |
| 7 | 60 | 65.3 | 65 | 63.5 | 53.1 | | |
| 8 | 59 | 58.5 | 68.5 | 66.5 | 52.7 | | |
| 9 | 56.9 | 62 | 67.6 | 63.9 | 54.6 | | |
| 10 | 59.2 | 59.3 | 65.1 | 64.9 | 63.4 | | |
| N | 10 | 10 | 10 | 10 | 10 | 5 | 5 |
| Mean | 59.2 | 60.8 | 67.0 | 63.9 | 55.1 | 53.3 | 53.4 |
| Dev. | 1.5 | 3.0 | 1.3 | 1.4 | 3.1 | 0.6 | 0.5 |

B. Seal Strength Testing Apparatus and Procedures

The product Codes and commercial products described above were tested for seal strength in a 180-degree peel adhesion test as follows.

1. Requirements

A commercially available Constant-Rate-of-Extension (CRE) tensile tester is used. Appropriate CRE testers and associated computer-based acquisition and frame control systems are available from Instron Corp. of Canton Mass., USA. or MTS Systems Corp of Research Triange Park, N.C., USA. An appropriate load cell is used or the selected CRE tester. It is recommended that the load cell be used in which the majority of the peak load results fall between 10% and 90% of the capacity of the load cell.

2. Verification and Calibration

The CRE tester is calibrated to national standards prior to testing. The following verification or calibration documents may be used:

ICP 7013, Constant-Rate-of-Extension (CRE) Tensile Testing Frame

ICP 7029, Constant-Rate-of-Extension (CRE) Tensile Tester Load Cells

3. Preparation
Verify appropriate load cell is in the tester and refer to manufacturer's specifications for load cell warm up and conditioning.
Ensure air pressure to operate grips is set at 414 kPa (60 psi).
Ensure the appropriate grips are installed in the tensile tester and that the grips and grip faces are free of build-up, dents, or other damage.
Turn on computer and follow software menu selections.
Follow instructions for calibrating the load cell for the tensile tester being used.

Verify the tensile tester parameters meet the following specifications:

| Crosshead Speed: | 508 +/− 10.2 | mm/min |
|---|---|---|
| Gage Length: | 50.8 +/− 1 | mm |
| Load Units: | Grams-force (gf) | |
| Run to Elongation: | 33 | mm |
| Slack Preload: | 50 | gf |
| Number of Cycles: | 1 | |
| Start Measurement: | 0 | mm |
| End of Measurement: | 15.2 +/− 1 | mm |

4. Test Specimen
Cut a slit along bottom of pouch and remove pad through the slit without disturbing sealed flap component of the pouch.
If pad is adhered to pouch, leave the pad in the pouch.
If flap is sealed to pouch between the lateral sides of the pouch, break adhesive seal between the sealed lateral sides of the pouch.
Mark along the edge of the flap.

5. Procedure
Center pouch in the grips with the flap in the upper grip and the bottom of the pouch in the lower grip. Do not clamp the pouch at an angle. Do not initiate peel when placing specimen in the grips.
Start the crosshead.
Record the peak load and the average load
After the cycle has finished, carefully remove the specimen.
Return the crosshead to its start position.
Repeat the procedure for each additional specimen

6. Results

Average Load (g_{f})

| | | | | | | Good |
|---|---|---|---|---|---|---|
| | | | | Kotex ® | Uni-Charm | Feel NW |
| | Code | Code | Code | Film | NW Pouch | Pouch |
| | A | B | C | Pouch | (Korean) | (Japan) |
| 1 | 28.4 | 40.6 | 175.0 | 433.9 | 69.2 | 150.4 |
| 2 | 42.9 | 40.8 | 390.9 | 238.4 | 57.0 | 197.7 |
| 3 | 86.9 | 40.6 | 179.4 | 532.2 | 58.1 | 143.2 |
| 4 | 50.0 | 60.2 | 222.7 | 435.5 | 73.3 | 158.1 |
| 5 | 32.0 | 60.1 | 205.2 | 189.8 | 57.9 | 186.4 |
| 6 | 40.1 | 48.4 | 346.0 | 356.6 | | 151.6 |
| 7 | 32.9 | 55.8 | 242.4 | 307.4 | | 119.8 |
| 8 | 29.9 | 54.7 | 206.9 | 496.4 | | 134.1 |
| 9 | 30.7 | 58.5 | 297.1 | 616.0 | | 148.5 |
| 10 | 36.3 | 52.3 | 173.6 | 397.2 | | 161.2 |
| N | 10 | 10 | 10 | 10 | 5 | 10 |
| Mean | 41.0 | 51.2 | 243.9 | 400.3 | 63.1 | 155.1 |
| Dev. | 17.5 | 8.1 | 76.0 | 131.9 | 7.6 | 23.0 |

Peak Load (g_{f})

| | | | | | | Y-K |
|---|---|---|---|---|---|---|
| | | | | | | K rean |
| | | | | K tex | Jap. Uni- | Good |
| | Code | C de | Code | Film | Charm NW | Feel NW |
| | A | B | C | Pouch | Pouch | Pouch |
| 1 | 54.5 | 66.592 | 308.1 | 750.0 | 113.2 | 633.6 |
| 2 | 80.1 | 67.704 | 961.8 | 616.3 | 126.7 | 798.7 |
| 3 | 272.2 | 66.966 | 480.6 | 1086.7 | 113.4 | 596.4 |
| 4 | 111.4 | 147.896 | 396.8 | 822.6 | 137.5 | 759.5 |
| 5 | 58.5 | 107.657 | 420.1 | 317.0 | 120.5 | 761.1 |
| 6 | 67.9 | 81.731 | 507.0 | 739.4 | | 642.3 |
| 7 | 100.7 | 90.179 | 405.0 | 686.7 | | 635.1 |
| 8 | 60.5 | 142.863 | 368.2 | 1123.5 | | 629.4 |
| 9 | 64.7 | 201.621 | 721.2 | 928.9 | | 624.3 |
| 10 | 62.8 | 99.712 | 430.0 | 759.8 | | 751.3 |
| N | 10 | 10 | 10 | 10 | 5 | 10 |
| Mean | 93.328 | 107.292 | 499.871 | 783.1 | 122.260 | 683.179 |
| Dev. | 65.603 | 44.304 | 196.569 | 233.0 | 10.202 | 74.695 |

The “Low Strength” pouch used in the tests recorded above resulted in a mean average load strength of 41 grams-force. This value is within the desired range of about 20 grams-force to about 60 grams-force. It should be understood that the tests results are for particular embodiments within the claimed range and are not reflective of the entire range. It is believed that seal strength values of between 20 grams-force and the tested sample value of 41 grams-force will also perform adequately in pouches according to the invention and are within the scope and spirit of the invention.

It should be appreciated by those skilled in the art that various modifications and variations can be made to the embodiments of the absorbent article described herein without departing from the scope and spirit of the invention as set forth in the appended claims and equivalents thereof.

1. An individually wrapped absorbent article package, comprising:
an absorbent article;
a wrapper material folded into a pouch, said absorbent article disposed within said pouch;
said wrapper material defining an opening flap when folded into said pouch;
said wrapper material comprising a nonwoven material in at least a border region along lateral sides of said pouch, said border region of nonwoven material defining a seal zone along said lateral sides of said pouch;
said opening flap having lateral sides sealed with opening flap seals to said pouch lateral sides along said seal zone such that a user opens said pouch by separating said opening flap seals; and
wherein said opening flap seals have a seal strength of between about 20 grams-force and about 60 grams-force and an opening noise level of less than about 60 db.

2. The package as in claim 1, wherein said opening flap seals are provided between said lateral sides of said flap and lateral side edges a back panel of said pouch.

3. The package as in claim 2, wherein said opening flap extends at least partially over a front panel of said pouch, said opening flap seals also provided at least partially with lateral side edges of said front panel.

4. The package as in claim 1, wherein said wrapper material is formed substantially entirely of said nonwoven material.

5. The package as in claim 1, wherein said wrapper material comprises a first sheet of said nonwoven material and an additional sheet of a film material attached thereto such that said nonwoven material defines said border region, said film material sheet having a configuration so as to generally line an interior of said pouch while leaving at least a portion of said nonwoven material exposed upon said wrapper material being folded into said pouch.

6. The package as in claim 5, wherein said film material sheet is comprises holes defined therethrough, said nonwoven material exposed by said holes.

7. The package as in claim 5, wherein said film material sheet is laminated to said nonwoven material sheet.

8. The package as in claim 5, wherein said border region is defined along lateral sides of said wrapper material, said film material sheet having a length generally equal to a length of said nonwoven material sheet.

9. The package as in claim 5, wherein said film material sheet is generally fluid-impervious.

10. The package as in claim 5, wherein said film material sheet is vapor permeable.

11. The package as in claim 5, wherein said exposed portions of said nonwoven material are defined adjacent to said seal.

12. The package as in claim 1, wherein said border region is defined along lateral sides of said wrapper material and extends laterally inward of said seal zone.

13. The package as in claim 1, wherein said wrapper material is folded at a first fold axis such that a first end of said wrapper material is folded towards a second opposite end to define a portion of said pouch, said wrapper material folded at a second fold axis such that said second end is folded towards said first fold axis and over said first end to define said opening flap.

14. The package as in claim 1, wherein said opening flap comprises a leading edge that is unsealed relative to said pouch between said sealed lateral sides of said pouch.

15. The package as in claim 1, wherein said absorbent article is one of a sanitary pad, panty liner, incontinence pad, and tampon.

16. The package as in claim 1, wherein said wrapper material comprises a first sheet of said nonwoven material and an additional sheet of a film material attached thereto such that said nonwoven material defines said border region, said film material sheet being air permeable.

17. An individually wrapped feminine care absorbent article package, comprising:
an absorbent article;
a wrapper material folded into a pouch, said absorbent article disposed within said pouch;
said pouch having at least one sealed seam along a side thereof that is opened by a consumer to retrieve said absorbent article;
said wrapper material comprising a nonwoven material at least along edges thereof defining said side when said wrapper material is folded into said pouch;
said sealed seam defined by sealed facing regions of a nonwoven material; and
wherein said sealed seam has a seal strength of between about 20 grams-force and about 60 grams-force and an opening noise level of less than about 60 db.

18. The package as in claim 17, wherein said sealed seam is thermally sealed.

19. The package as in claim 17, wherein said sealed seam is defined along at least one lateral side of said pouch.

20. The package as in claim 17, wherein said wrapper material is formed substantially of said nonwoven material.

21. The package as in claim 17, wherein said wrapper material comprises a border region of said nonwoven material along lateral sides thereof.

22. The package as in claim 21, wherein said wrapper material comprises a film material layer bordered by said border region of nonwoven material.

23. The package as in claim 22, wherein said wrapper material comprises a first sheet of said nonwoven material and a second sheet of said film material attached to said nonwoven material sheet.

24. The package as in claim 17, wherein said pouch comprises an opening flap having an edge coincident with said sealed seam.

25. An individually wrapped absorbent article package, comprising:
an absorbent article;
a wrapper material folded into a pouch, said absorbent article disposed within said pouch;
an openable seal provided in said wrapper material wherein a user gains access to said absorbent article by opening said seal;
said wrapper material comprising a nonwoven material in at least a region along said seal; and
wherein said seal has a seal strength of between about 20 grams-force and about 60 grams-force.

26. The package as in claim 25, wherein said seal has an opening noise level of less than about 60 db.

27. The package as in claim 25, wherein said wrapper material defines an opening flap when folded into said pouch, said seal defined by lateral sides of said opening flap sealed to lateral sides of said pouch.

28. The package as in claim 25, wherein said seal is defined along at least one side of said pouch.

29. The package as in claim 28, wherein said seal is defined between opposite layers of said wrapper material.

30. The package as in claim 28, wherein said seal is defined at least partially in a single layer of said wrapper material.
